(19) United States
(12) Patent Application Publication
(10) Pub. No.: US 2018/0140277 A1
(43) Pub. Date: May 24, 2018

(54) TRANSDUCER ADAPTERS FOR ALLOWING MULTIPLE MODES OF ULTRASOUND IMAGING USING A SINGLE ULTRASOUND TRANSDUCER

(71) Applicant: Clarius Mobile Health Corp., Burnaby (CA)

(72) Inventors: Laurent PELISSIER, North Vancouver (CA); Kris DICKIE, Vancouver (CA); Binda ZHANG, Surrey (CA); Nishant UNIYAL, Vancouver (CA)

(21) Appl. No.: 15/630,916

(22) Filed: Jun. 22, 2017

Related U.S. Application Data
(60) Provisional application No. 62/424,152, filed on Nov. 18, 2016.

(51) Int. Cl. A61B 8/00; A61B 8/08

(52) U.S. Cl. CPC A61B 8/4483; A61B 8/4272; A61B 8/0883; A61B 8/5207

(57) ABSTRACT

The present embodiments relate generally to ultrasound imaging systems. The ultrasound imaging systems may include an ultrasound imaging transducer operable to acquire ultrasound image data and a transducer adapter configured to detachably couple to the ultrasound imaging transducer. The transducer adapter may provide a different footprint from the ultrasound imaging transducer native footprint to enable the ultrasound imaging transducer adapter to acquire ultrasound image data in a substantially similar way to ultrasound transducers with different transducer geometry. The ultrasound imaging transducer may include a sensor for detecting an attachment state of the transducer adapter. The ultrasound imaging transducer may modify one or more imaging parameters when the transducer is attached to the ultrasound imaging transducer.

### CROSS-REFERENCE TO RELATED APPLICATIONS

This application claims the benefit of U.S. Provisional Patent Application No. 62/424,152 entitled “TRANSDUCER ADAPTERS FOR ALLOWING MULTIPLE MODES OF ULTRASOUND IMAGING USING A SINGLE ULTRASOUND TRANSDUCER” filed on Nov. 18, 2016, which is incorporated by reference it its entirety in this disclosure.

### FIELD

The present disclosure relates generally to ultrasound imaging, and particularly, adapters for ultrasound transducers.

### BACKGROUND

Traditional ultrasound systems are typically used with a number of different ultrasound probes that are designed to image different parts of the body. These different types of ultrasound probes have different transducer element configurations that make them suitable for imaging different parts of the body.

For example, a phased-array probe typically has a small footprint that allows the probe to be positioned on parts of the body that have constricted space (e.g., in the intercostal space in between a patient's ribs). Since imaging the heart is a common use for this type of probe, it is also called a cardiac probe.

In another example, a sequential curvilinear-array probe (also called a convex or curved probe) contains a larger footprint, with the transducer elements on the probe being positioned on a curve to provide a wide field of view. This configuration makes the curvilinear array probe suitable for imaging the abdomen.

In a further example, a sequential linear array probe may similarly have a wider footprint than that of a phased-array probe. Unlike a cardiac probe or a curvilinear probe, the linear probe directs parallel ultrasound signals from its linear transducer array so as to provide substantially similar lateral resolution in the near and far field. Linear array probes also typically have a shorter elevation length, so as to provide a finer slice thickness resolution.

Using different probes to examine different parts of the body is inconvenient. For example, in examinations performed in an emergency medicine context (e.g., during a Focused Assessment with Sonography in Trauma (FAST) examination), it is desirable to quickly examine multiple internal organs to arrive at a quick medical assessment. The time delay caused by the switching of probes may delay the performance of such examinations.

There is thus a need for improved methods and apparatus for imaging different areas of a patient using the same ultrasound probe.

### BRIEF DESCRIPTION OF THE DRAWINGS

Non-limiting examples of various embodiments of the present disclosure will next be described in relation to the drawings, in which:

FIG. 1 shows a curvilinear ultrasound probe with example physical transducer adapters, in accordance with at least one embodiment of the present invention;

FIG. 2 shows the ultrasound probe of FIG. 1 being provided with a cardiac adapter, in accordance with at least one embodiment of the present invention;

FIG. 3 shows the time delays and apertures used to perform beamforming during operation of an ultrasound probe with a cardiac adapter attached, in accordance with at least one embodiment of the present invention;

FIG. 4 shows a cross-sectional view of the ultrasound probe's transducer array and the cardiac adapter shown in FIG. 2, in accordance with at least one embodiment of the present invention;

FIG. 5 shows the ultrasound probe of FIG. 1 being provided with a linear adapter, in accordance with at least one embodiment of the present invention;

FIG. 6 shows the time delays and apertures used to perform beamforming during operation of the ultrasound probe with the linear adapter attached, in accordance with at least one embodiment of the present invention;

FIG. 7 shows a cross-sectional view of the ultrasound probe's transducer array and the linear adapter shown in FIG. 5, in accordance with at least one embodiment of the present invention;

FIG. 8 shows a bottom view of the ultrasound probe and linear adapter of FIG. 5, in accordance with at least one embodiment of the present invention;

FIG. 9 shows a curvilinear ultrasound probe with example physical transducer adapters, in accordance with at least one embodiment of the present invention;

FIG. 10 shows an interior perspective views of the cardiac adapter and linear adapter of FIG. 9, in accordance with at least one embodiment of the present invention;

FIG. 11 shows a close-up perspective view of a pass-through volume component for the linear adapter in relation to an ultrasound probe, in accordance with at least one embodiment of the present invention;

FIG. 12 is a flowchart diagram showing steps of a method for generating ultrasound images with an ultrasound imaging transducer and transducer adapter, in accordance with at least one embodiment of the present invention;

FIG. 13 shows an interior perspective views of an alternative embodiment of the cardiac adapter and linear adapter, in accordance with at least one embodiment of the present invention;

FIG. 14 shows a cross-sectional view of the cardiac adapter and linear adapter of FIG. 13, in accordance with at least one embodiment of the present invention; and

FIG. 15 shows the different imaging configurations of the ultrasound imaging probe and example cardiac and linear transducer adapters, in accordance with at least one embodiment of the present invention.

### DETAILED DESCRIPTION

In a first broad aspect of the present disclosure, there is provided an ultrasound imaging assembly, including an ultrasound imaging transducer having transducer elements configured in a curved geometry; and a transducer adapter for coupling to the ultrasound imaging transducer, wherein the transducer adapter includes a pass-through volume for permitting ultrasound energy to be transmitted through the transducer adapter, the pass-through volume including: a proximal surface for mating to the transducer, the proximal surface having a curvature corresponding to the curved geometry of the transducer elements; and a distal surface at a distal end of the transducer assembly, the distal surface having a geometry that is substantially planar.

In some embodiments, the transducer adapter is configured to releasably couple to the ultrasound imaging transducer.

In some embodiments, the ultrasound imaging transducer assembly is configured to determine an adapter type of the transducer adapter. In some embodiments, the ultrasound imaging transducer assembly includes a sensor for determining the adapter type of the transducer adapter.

In some embodiments, the transducer adapter includes a radio frequency identification (RFID) tag and the sensor includes a radio frequency identification (RFID) sensor.

In some embodiments, the transducer adapter includes an image signature feature readable by the ultrasound imaging transducer to determine when the transducer adapter is coupled to the ultrasound imaging transducer.

In some embodiments, the pass-through volume includes at least one of agar, agarose, Aqualene™, silicone, polyvinyl alcohol, polyvinyl alcohol gel, polyacrylamide gel, open porosity foam, gelatin gel, oil gel, polyurethane gel, epoxy plastisol, silicon rubber, swollen segmented polyurethane gel (S-SPUG), urethane polymer, tofu, magnesium silicate, and Zerdine™.

In some embodiments, the ultrasound transducer elements have a first elevational length and the proximal surface has a second elevational length, different from the first elevational length.

In another broad aspect of the present disclosure, there is provided a method of generating ultrasound images with an ultrasound imaging transducer, the ultrasound imaging transducer having a native footprint, the method including: coupling a transducer adapter to the ultrasound imaging transducer, wherein the transducer adapter has a proximal end configured to mate to the ultrasound imaging transducer and a distal end that defines a contact surface, wherein the contact surface corresponds to an adapted footprint different from the native footprint; modifying at least one imaging parameter so that, during imaging, ultrasound signals emitted from the ultrasound imaging transducer travels through the transducer adapter and exits the contact surface, and the exiting ultrasound signals have one or more characteristics substantially similar to other ultrasound signals emitted from another ultrasound imaging transducer having the adapted footprint as its native footprint; and generating the ultrasound images with the modified at least one imaging parameter.

In some embodiments, after the transducer adapter is coupled, the modified at least one imaging parameter causes at least one of the ultrasound signals emitted from the ultrasound imaging transducer to be steered in a direction away from normal to the native footprint of the ultrasound imaging transducer.

In some embodiments, the ultrasound imaging transducer emits non-steered ultrasound signals during imaging prior to the transducer adapter being coupled.

In some embodiments, the modified at least one imaging parameter includes at least one of time delay and aperture, to cause at least one of the ultrasound signals to be steered in the direction away from normal to the native footprint of the ultrasound imaging transducer.

In some embodiments, the modified at least one ultrasound imaging parameter includes at least one of time delay, sequence, steering angle, transmit aperture size, transmit aperture location, receive aperture size, receive aperture location, and image zero point.

In some embodiments, the adapted footprint includes a cardiac footprint, and the exiting ultrasound signals are steered in respective different directions so that a substantial portion of a sector image is generated.

In some embodiments, the adapted footprint includes a linear footprint, and the steered at least one of the ultrasound signals emitted from the ultrasound imaging transducer results in the exiting ultrasound signals being projected orthogonally to the contact surface.

In some embodiments, the exiting ultrasound signals are parallel so that a rectangular image is generated.

In some embodiments, after coupling the transducer adapter, the method includes determining an adapter type of the transducer adapter, wherein the adapter type is based on at least one of a geometry of: the proximal end configured to mate to the ultrasound imaging transducer, and a geometry of the contact surface.

In some embodiments, the modifying of the at least one imaging parameter is based at least in part on the adapter type.

In some embodiments, the ultrasound imaging transducer includes a sensor and determining the adapter type of the transducer adapter includes sensing the adapter type of the transducer adapter using the sensor.

In some embodiments, an image signature feature is provided on the transducer adapter that is readable when the transducer adapter is coupled to ultrasound imaging transducer, and determining the adapter type of the transducer adapter includes: generating an ultrasound image, and identifying the image signature feature on the ultrasound image to determine the adapter type of the transducer adapter.

In some embodiments, determining the adapter type of the transducer adapter includes: receiving input at the ultrasound imaging transducer, the input indicating the adapter type of the transducer adapter. For example, the input may be provided via a suitable user interface on a computing device that controls and/or communicates with the ultrasound imaging transducer.

In some embodiments, the adapter type includes at least one of a cardiac phased array contact surface and a linear array contact surface.

In another broad aspect of the present disclosure, there is provided a transducer adapter for coupling to an ultrasound imaging transducer with a native footprint, the transducer adapter including: a proximal end configured to mate to the ultrasound imaging transducer; and a distal end that defines a contact surface, wherein the contact surface corresponds to an adapted footprint different from the native footprint; wherein when the transducer is coupled to the ultrasound imaging transducer, ultrasound signals emitted from the ultrasound imaging transducer travels through the transducer adapter and exits the contact surface, and the exiting ultrasound signals have one or more characteristics that are substantially similar to ultrasound signals emitted from another ultrasound imaging transducer having the adapted footprint as its native footprint.

In some embodiments, the transducer adapter includes a pass-through volume for permitting the ultrasound signals to be transmitted through the transducer adapter.

In some embodiments, the pass-through volume includes at least one of agar, agarose, Aqualene™, silicone, polyvinyl alcohol, polyvinyl alcohol gel, polyacrylamide gel, open porosity foam, gelatin gel, oil gel, polyurethane gel, epoxy plastisol, silicon rubber, swollen segmented polyurethane gel (S-SPUG), urethane polymer, tofu, magnesium silicate, and Zerdine™.

In some embodiments, the transducer adapter includes an image signature feature readable by the ultrasound imaging transducer when the transducer adapter is couple to the ultrasound imaging transducer.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, certain steps, signals, protocols, software, hardware, networking infrastructure, circuits, structures, techniques, well-known methods, procedures and components have not been described or shown in detail in order not to obscure the embodiments generally described herein.

Furthermore, this description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

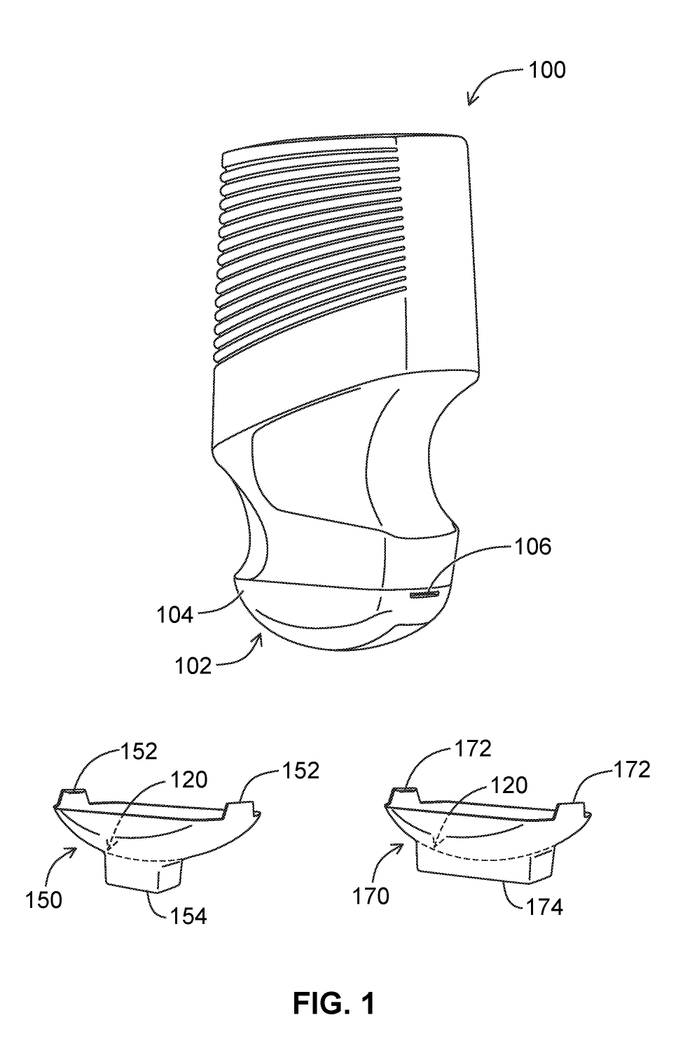

Referring to FIG. 1, shown there generally as 100 is a curvilinear ultrasound probe with example physical transducer adapters, in accordance with at least one embodiment of the present invention. As illustrated, the curvilinear probe 100 is provided in the form of a handheld wireless scanner that may be configured to communicate with an external wireless computing device containing a display (not shown). However, in other embodiments, the ultrasound probe can be provided in other forms. For example, the physical adapters may be used with an ultrasound probe that can be attached via a cord to a separate ultrasound machine. While generally referred herein as ultrasound probe 100, the ultrasound probe 100 may in some instances additionally or alternatively be referred to as an ultrasound imaging transducer herein.

Different hardware adapters 150, 170 may be attached to the probe head 102. The hardware adapters 150, 170 may each have a surface 120 that is constructed so that it conforms to the to the exterior surface of the probe head 102. In the illustrated embodiments, the hardware adapters 150, 170 each have a cavity with such mating surface 120 provided on the interior of the cavity. The housing of the adapters 150, 170 may be made of any suitable material. For example, in some embodiments, the housing of the adapters 150, 170 may be made of molded plastic. When the different adapters 150, 170 are each attached the probe head 102, the ultrasound signals emitted from the probe head 102 may be directed in a manner that allows for their transmission through a pass-through volume (discussed below) of the adapters 150, 170, so that the signals exit from the non-attached surfaces 154, 174 of the adapters 150, 170 respectively. Echoes corresponding to the transmitted ultrasound signals can be received via the same pass-through volumes at the probe head 102.

To allow the ultrasound signals to travel through the adapters 150, 170, the adapters 150, 170 may be constructed so that the pass-through volumes through which the ultrasound signals travel are provided with an acoustically transparent material. For example, the acoustically transparent material may be a phantom-like material or an epoxy (e.g., an epoxy material that may be similar to what is be provided on the surface of the probe head 102 itself. In various embodiments, the acoustically transparent material may additionally or alternatively be made of one or more of agar, agarose, Aqualene™, silicone, polyvinyl alcohol, polyvinyl alcohol gel, polyacrylamide gel, open porosity foam, gelatin gel, oil gel, polyurethane gel, epoxy plastisol, silicon rubber, swollen segmented polyurethane gel (S-SPUG), urethane polymer, tofu, magnesium silicate, and/or Zerdine™. In an example embodiment, Aqualene™ M320 from Olympus Corporation may used as the material for the pass-through volume. The acoustically transparent material of the pass-through volumes may extend from the attached surfaces 120 to the respective non-attached surfaces 154, 174 of the adapters 150, 170.

The non-attached surfaces 154, 174 may be placed against the skin of a patient to perform examinations. By providing acoustically transparent material at the interface of the probe head 102 and the attached surfaces 120 of the adapters 150, 170, and also through the interior pass-through volumes of the adapters 150, 170, there may be minimal loss of acoustic energy due to reflection or refraction prior to the ultrasound energy entering patient tissue. In some embodiments, a coupling agent (e.g., ultrasound gel) may be placed on the attached surfaces 120 of the adapters 150, 170 prior to attaching the adapters to the probe head 102. In various embodiments, the non-attached surfaces 154, 174 of the adapters 150, 170 may also be provided with an acoustically transparent material so that the ultrasound signals can be emitted therefrom and received thereat with minimal loss of acoustic energy. While the term pass-through volume is generally used herein, the same element may generally be referred to as the “lens” of a transducer adapter 150, 170 in various instances herein.

The element described herein generally as attached surface 120 may in certain instances also be referred to as the proximal surface of the transducer adapters 150, 170. Likewise, the element described herein as non-attached surfaces 154, 174 may also be referred to as the distal surface. Further, since the attached surface 120 may mate with the transducer adapters 150, 170 when a transducer adapter 150, 170 is attached, the attached surface 120 may additionally or alternatively be considered a mating surface. Likewise, since the non-attached surfaces 154, 174 may contact an object (e.g., skin) during imaging when the adapters 150, 170 are attached, the non-attached surfaces 154, 174 may additionally or alternatively be considered a contact surface. As discussed herein, the mating surface 120 is generally described as forming an acoustic coupling with an exterior surface of the transducer where ultrasound signals are emitted/received. This coupling may be formed via direct or indirect physical contact. For example, in various embodiments, the mating surface 120 and the transducer may not form physical contact. Instead, the acoustic coupling may be formed indirectly through a suitable acoustic coupling agent such as ultrasound gel.

In various embodiments, the nosepiece 104 holding the transducer array on the ultrasound probe 100 may be configured with a mechanism to allow for attachment of the adapters 150, 170. For example, the nosepiece 104 may be provided with a number of slots or grooves 106 for receiving corresponding clamps or clips 152, 172 on the adapters 150, 170. The clips 152, 172 may slide over the body of the nosepiece 104 to engage the grooves 106. The adapters 150, 170 may be removed, for example, by physically pulling on the adapters 150, 170 so that clips 152, 172 disengage. Other methods of attaching the adapters 150, 170 to the nosepiece 104 may be possible. For example, a slidable latch may be provided in some embodiments. Additionally or alternatively, the adapters 150, 170 may be secured on the nosepiece 104 via a friction fit (e.g., without grooves 106 or clamps 152, 172).

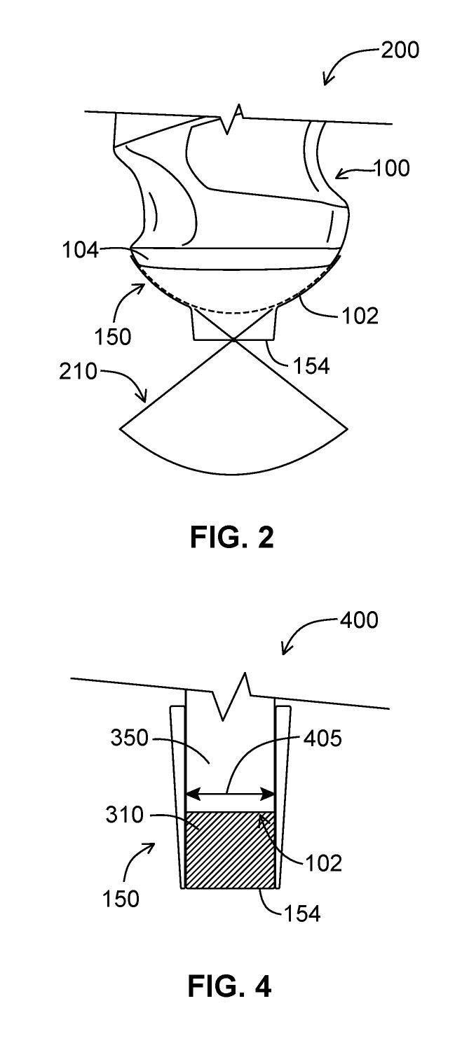

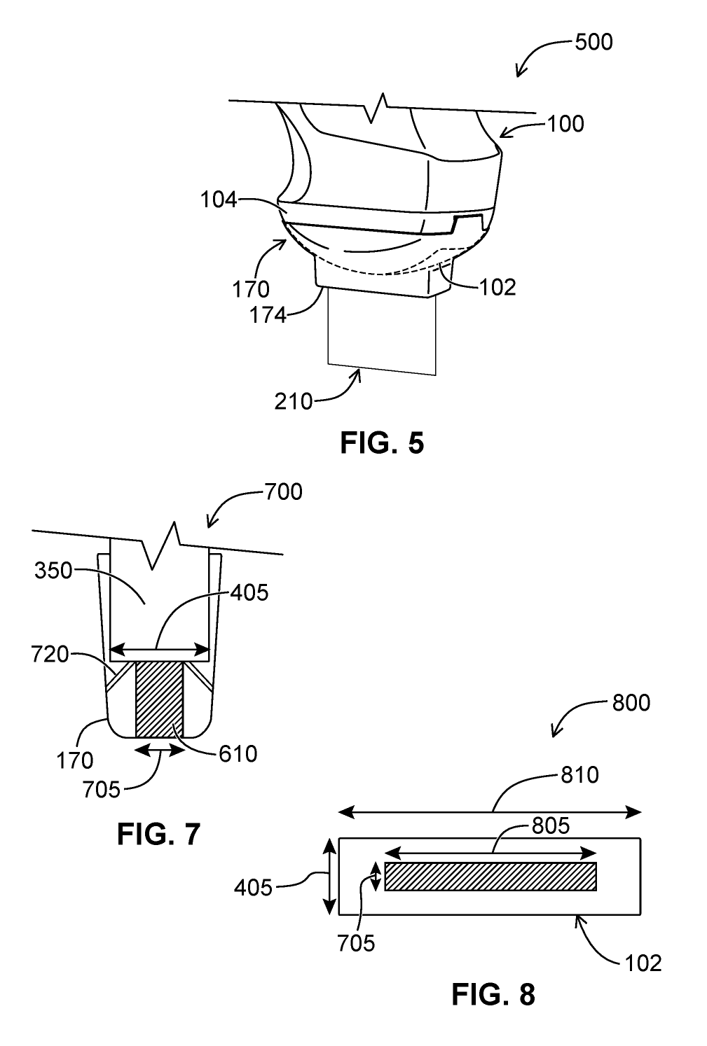

Two example transducer adapters are shown in FIG. 1: a cardiac adapter 150 and linear adapter 170. The operation of the cardiac adapter 150 is discussed below with respect to FIGS. 2-4. The operation of the linear adapter 170 is discussed below with respect to FIGS. 5-8. In the illustrated example embodiment, the transducer array provided on the probe head 102 is a curvilinear transducer array. However, in other embodiments, the principles discussed herein may be applied to configure ultrasound probes with other transducer array configurations to operate with hardware adapters to provide different ultrasound probe footprints and imaging capabilities.

The cardiac adapter 150 and linear adapter 170 may be considered two examples of transducer adapter types. Other adapter types may be defined based on the geometry of the attached surface 120 and non-attached surfaces of given the transducer adapters. As noted, these the attached surface 120 and non-attached surfaces discussed herein may also be referred to as the mating surface and contact surface, respectively.

Referring to FIG. 2, shown there generally as 200 is the ultrasound probe of FIG. 1 being provided with a cardiac adapter 150, in accordance with at least one embodiment of the present invention. As illustrated, the adapter 150 is attached to the nose piece 104 of the curvilinear probe 100 of FIG. 1, and the probe head 102 (shown in dotted outline) is mated to the attached surface 120 of the cardiac adapter 150. The non-attached surface 154 of the adapter 150 is shaped like a traditional phased-array cardiac probe. Specifically, the non-attached surface 154 is provided with a smaller footprint that allows for better insertion of the non-attached surface 154 in the intercostal space. The smaller footprint may allow the lowering of the non-attached surface 154 into the intercostal space so that the zero point of the image is slightly lower (e.g., by 5 millimeters). This may allow for improved imaging of the heart. For ease of reference, the cardiac adapter 150 is referred to herein as the “cardiac” adapter. However, it will be understood by persons skilled in the art that the ultrasound probe 100 when operating with such adapter 150 attached may provide phased-array imaging that is usable in non-cardiac medical examinations.

Traditional phased-array cardiac probes have a small footprint and thus a small transducer array provided on its probe head. To provide a sufficiently large field of view, these probes may repeatedly activate the transducer elements and steer ultrasound signals in a variety of directions. This results in a sector image. When the cardiac adapter 150 is attached to the ultrasound probe 100 in the present embodiments, a similar set of sweeping ultrasound signals may be transmitted from the relatively smaller footprint of the non-attached surface 154 of the cardiac adapter 150. To accomplish this, the ultrasound sequence in which the elements of the transducer array is activated may be altered. For example, different subsets of the transducer elements may be selectively activated and steered so as to project the ultrasound signals 210 in the manner shown in FIG. 2 to obtain a sector image with its apex at the non-attached surface 154 of the cardiac adapter 150. Additional teachings related to how subsets of the transducer elements within a transducer array may be activated and selectively steered are discussed in Applicant's U.S. patent application Ser. No. 15/207,203 (referred to herein as “Applicant's Virtual Phased-array Application”), which is hereby incorporated by reference in its entirety.

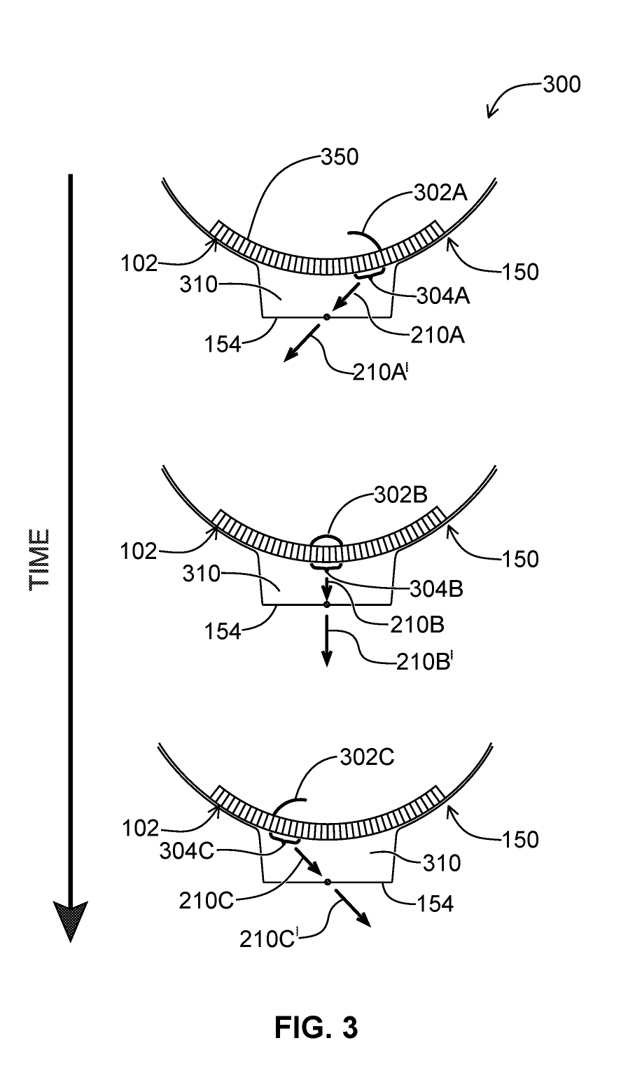

Referring to FIG. 3, shown there generally as 300 are the time delays and apertures used to perform beamforming when the transducer array of the curvilinear probe 100 is activated with the cardiac adapter 150 attached thereto, in accordance with at least one embodiment of the present invention. FIG. 3 shows a simplified view of a transducer head 102 with its constituent transducer elements 350 and how they are pulsed at three example points in time during generation of an ultrasound image when the cardiac adapter 150 is attached.

Beamforming involves applying a time delay to when adjacent transducer elements 350 are pulsed so that the interference pattern generated by ultrasound signals 210 form a beam when projected. By varying the time delay and sequence in which the transducer elements 350 within a selected subset of the transducer elements are pulsed, the beam can be steered.

To generate a sector image from the non-attached surface 154 of the cardiac adapter 150, ultrasound beams are transmitted from selected groups of adjacent transducer elements 350 across a middle portion of the transducer array. These ultrasound beams result in the formation of scanlines that collectively generate the ultrasound image. The position of the transducer elements 350 on the probe head 102 where the ultrasound signals 210 get generated may be called the “aperture”. As will be understood by persons skilled in the art, ultrasound operation may involve a transmit aperture and a receive aperture. The transmit aperture refers to the transducer elements 350 that are activated when the ultrasound signals 210 are generated, and the receive aperture refers to the transducer elements 350 that receive echo energy in response. The two apertures may be different such that they include different groups of transducer elements 350. Unless specifically indicated, the term “aperture” refers to the transmit aperture herein.

At the first point in time, the aperture 304A is on a portion of the transducer array that is right of the center point of the transducer array, so that a group of adjacent transducer elements 350 there are pulsed. This group of adjacent transducer elements 350 are pulsed according to a time delay 302A. The time delay 302A is illustrated as an arc that represents the sequence of activation when the transducers elements 304A are pulsed. As shown, the time delay 302A shown has the rightmost transducer elements 350 within the aperture 304A being activated first and then progressively shifting to the left of the aperture 304A in the sequence and manner represented by the time delay 302A. The time delay 302A will cause the ultrasound signal 210A to be directed to the left. Such an ultrasound signal 210A may be projected through the pass-through volume 310 (as may be filled with epoxy or other acoustically transparent material) of the cardiac adapter 150 so as to exit the cardiac adapter 150 through the apex of the resultant sector image (shown as a dot in FIG. 3) at the non-attached surface 154 in the direction shown (e.g., as ultrasound signal 210A′).

At the second point in time, the aperture 304B is in the center portion of the transducer array. The time delay 302B that is applied starts with the outermost transducer elements 350 of the aperture 304B being pulsed first, and then transducer elements 350 towards the center of the aperture 304B are progressively pulsed. This type of time delay 302B may generate an ultrasound beam 210B that focuses in a direction orthogonal to the surface of the probe head 102. The ultrasound beam 210B may travel through the pass-through volume 310 and exit the cardiac adapter 150 through the apex of the resultant sector image at the non-attached surface 154 (e.g., as ultrasound signal 210B′).

At the third point in time, the aperture 304C is on a portion of the transducer array that is left of the center point of the transducer array, so that a group of adjacent transducer elements 350 there are pulsed. This group of adjacent transducer elements 350 are pulsed according to a time delay 302C. As shown, the time delay 302C shown has the leftmost transducer elements 350 within the aperture 304C being activated first and then progressively shifting to the right of the aperture 304C in the sequence and manner represented by the time delay 302C. The time delay 302C will cause the ultrasound signal 210C to be directed to the right. Such an ultrasound signal 210C may be projected through the pass-through volume 310 of the cardiac adapter 150 so as to exit the cardiac adapter 150 through the apex of the resultant sector image at the non-attached surface 154 in the direction shown (e.g., as ultrasound signal 210C′).

By selectively identifying apertures 304A, 304B, 304C and activating them with respective time delays 302A, 302B, 302C, the distance between the probe head 102 and the non-attached surface 154 of the cardiac adapter 150 may be accounted for. This may allow the ultrasound signals emitted from the smaller footprint of the cardiac adapter 150's non-attached surface 154 to have characteristics that are substantially similar to ultrasound signals that are emitted from a separate dedicated small footprint phased-array transducer. For example, the ultrasound signals 210 exiting the contact surface of the cardiac adapter 150 can mimic the ultrasound signals emitted from a traditional phased-array transducer, and provide analogous ultrasound imaging without the need of a separate dedicated small footprint phased-array probe.

Referring to FIG. 4, shown there is a cross-sectional view of the transducer array of the ultrasound probe and cardiac adapter of FIG. 2, in accordance with at least one embodiment of the present invention. FIG. 4 shows a simplified view of a transducer element 350 in the transducer array, with its bottom surface corresponding to the probe head 102. For ease of illustration, the transducer element 350 is shown as being positioned adjacent the pass-through volume 310 (shown in hatched shading) of the cardiac adapter 150, without showing intermediate layers such as the matching layers or the acoustic lens. As illustrated, the transducer element 350 has an elevation length 405, and the corresponding portion of the attached surface 120 of the cardiac adapter 150 that mates with the transducer element 350 has a matching elevation length, so that the elevation/slice thickness resolution of the transducer element 350 can be maintained when ultrasound signals are emitted from the non-attached surface 154.

Referring to FIG. 5, shown there generally as 500 is the ultrasound probe of FIG. 1 being provided with a linear adapter, in accordance with at least one embodiment of the present invention. As illustrated, the linear adapter 170 is attached to the curvilinear probe 100 of FIG. 1, and the probe head 102 is mated to the attached surface 120 of the linear adapter 170. The linear adapter 170 is configured so that the footprint of its non-attached surface 174 is shaped like a traditional linear-array probe. When operating with the linear adapter 170 attached, the frequency of the ultrasound signals emitted may be lower than what is typically transmitted from a traditional linear ultrasound probe. However, the lower frequency may still be suitable for certain types of examinations (e.g., vascular). At the same time, in contrast to the curved configuration of the curvilinear transducer array, the flat footprint of the linear adapter 170 may allow for improved contact with the skin to perform such examinations.

Traditional linear-array probes may sequentially project parallel ultrasound signals across the transducer array, so as to produce a rectangular image. When the linear adapter 170 is attached to the ultrasound probe 100 in the present embodiments, a similar set of parallel ultrasound signals may be transmitted from the non-attached surface 174 of the linear adapter 170. To project a similar set of ultrasound signals from the linear footprint of the linear adapter 170, the time delays and sequence in which the elements of the transducer array are activated may be altered. For example, as illustrated, different subsets of the transducer elements may need to be activated so as to project the ultrasound signals 210 in the manner shown to obtain a rectangular image.

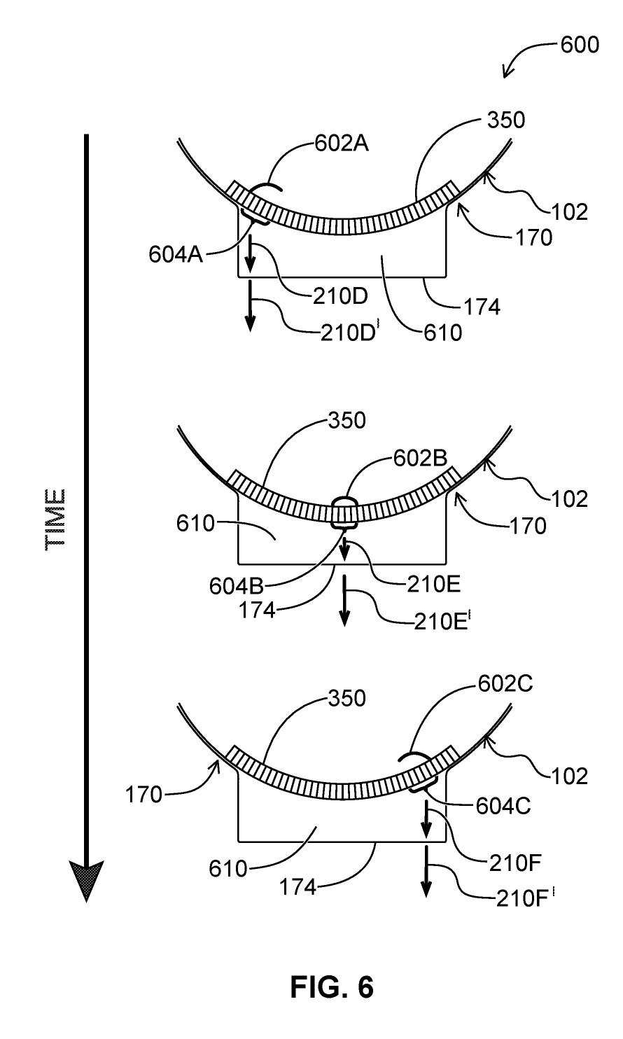

Referring to FIG. 6, shown there generally as 600 are the time delays and apertures used to perform beamforming when the transducer array of the curvilinear probe 100 is activated with the linear adapter 170 attached thereto, in accordance with at least one embodiment of the present invention. By varying the time delay and sequence in which the transducer elements 350 within a subset of the transducer elements are pulsed, the beams can be steered so as to provide ultrasound beams that are emitted from the non-attached surface 174 that mimic those typically emitted from a linear-array probe.

FIG. 6 shows a simplified view of the transducer array provided on probe head 102 with its constituent transducer elements 350 and how they are pulsed at three example points in time during generation of an ultrasound image when the linear adapter 170 is attached. To generate a rectangular image, ultrasound beams are transmitted from selected groups of adjacent transducer elements 350 sequentially and successively across a portion of the transducer array that excludes the peripheral transducer elements. These ultrasound beams result in the formation of parallel scanlines that collectively generate the rectangular ultrasound image.

At the first point in time, the aperture 604A is on a portion of the transducer array 102 that is proximately above the left edge of the non-attached surface 174 of the linear adapter 170, so that a group of adjacent transducer elements 350 there are pulsed. This group of adjacent transducer elements 350 are pulsed according to a time delay 602A. The time delay 602A is illustrated as an arc that represents the sequence of activation when the transducers elements 604A are pulsed. As shown, the time delay 602A shown has the leftmost transducer elements 350 within the aperture 604A being activated first and then progressively shifting to the right of the aperture 604A in the sequence and manner represented by the time delay 602A. The time delay 602A will cause the ultrasound signal 210D to be steered in a manner that is angled away from the azimuth/normal at aperture 604A. However, the signal 210D will be projected through the pass-through volume 610 (e.g., containing epoxy or other acoustically transparent material) of the linear adapter 170 so as to exit the linear adapter 170 in a direction that is orthogonal to the non-attached surface 174 (e.g., as ultrasound signal 210D′).

At the second point in time, the aperture 604B is in the center portion of the transducer array. Similar to the time delay 302B shown in FIG. 3, the time delay 602B that is applied starts with the outermost transducer elements 350 of the aperture 604B being pulsed first, and then transducer elements 350 towards the center of the aperture 604B are progressively pulsed. This type of time delay 602B may generate an ultrasound beam 210E that focuses in a direction orthogonal to the surface of the probe head 102. The ultrasound beam 210E may travel through the pass-through volume 610 and exit the linear adapter 170 in a manner that is also orthogonal to the non-attached surface 174 (e.g., as ultrasound signal 210E′).

At the third point in time, the aperture 604C is on a portion of the transducer head 102 that is proximately above the right edge of the non-attached surface 174 of the linear adapter 170. This group of adjacent transducer elements 350 are pulsed according to a time delay 602C. As shown, the time delay 602C has the rightmost transducer elements 350 within the aperture 604C being activated first and then progressively shifting to the left of the aperture 604C in the sequence and manner represented by the time delay 602C. The time delay 602C will cause the ultrasound signal 210F to be angled away from the azimuth at aperture 604C. However, the signal 210F will be projected through the pass-through volume 610 of the linear adapter 170 so as to exit the linear adapter 170 in a direction that is orthogonal to the non-attached surface 174 (e.g., as ultrasound signal 210F′).

Referring to FIG. 7, shown there generally as 700 is a cross-sectional view of the ultrasound probe 100's transducer array and the linear adapter shown in FIG. 5, in accordance with at least one embodiment of the present invention. FIG. 7 shows a simplified view similar to FIG. 4 discussed above. In addition to parallel ultrasound beams in the lateral direction, traditional linear ultrasound probes also typically have transducer elements with a shorter elevation length so as to provide finer elevation (also called slice thickness) resolution.

As in FIG. 4, the transducer element 350 shown in FIG. 7 has an elevation length 405. However, with the linear adapter 170 attached, the pass-through volume 610 of the adapter 170 has an elevation length 705 that is shorter than the elevation length 405 of the transducer elements on the probe head 102. This may allow for the ultrasound signals being emitted from the linear adapter 170 to be masked in the elevation direction, so as to provide an imaging slice thickness that is similar to that which is provided with traditional linear ultrasound probes.

Referring to FIG. 8, shown there generally as 800 is a bottom view of the ultrasound probe and linear adapter of FIG. 5, illustrating the masking of ultrasound signals achieved by the linear adapter, in accordance with at least one embodiment of the present invention. In FIG. 8, the width 810 and elevation length 405 of the transducer array for the curvilinear probe head 102 is shown. As illustrated, it can be seen that masking of the ultrasound signals typically transmitted from the transducer array of the curvilinear probe 100 is performed in multiple dimensions. For example, in the horizontal direction, it can be seen that the masking by the pass-through volume 610 may narrow the width of ultrasound signals that would typically be transmitted by the transducer array to the narrower width 805 of the non-attached surface 174 of the linear adapter 170. When this narrower width 805 is used with the modified ultrasound sequence discussed above with respect to FIG. 6, parallel ultrasound signals may be emitted from the non-attached surface 174 of the linear adapter 170 in a manner similar to that typically emitted from a traditional linear array probe. In the elevation length direction, it can be seen that the pass-through volume 610 also narrows the elevation length from the elevation length 405 of the transducer elements 350 to the narrower elevation length 705.

Various mechanisms may be used to accomplish the masking of ultrasound signals discussed herein. For example, the volume corresponding to the masked area (shown without hatched shading in FIG. 8) may be provided with material that absorbs and/or disperses ultrasound energy. Referring simultaneously to FIG. 7, in some embodiments, such material may be provided in any portion of the volume adjacent the pass-through volume 610. For example, such material may be provided as an air gap since air has low acoustic impedance and is highly absorbing. In various embodiments, other low acoustic impedance material can be used.

Additionally or alternatively, the linear adapter 170 may be provided with reflectors 720 adjacent the pass-through volume 610. By angling the reflectors 720 in a manner that reflect ultrasound energy away from the probe head 102, the ultrasound energy transmitted from the transducer element 350 that should be masked may be dispersed and only the desired ultrasound energy will travel through the pass-through volume 610 into the tissue and corresponding echoes will be received by the transducer element 350. In various embodiments, image analysis can be performed to identify the absorbed and/or dispersed signals caused by the air and/or reflectors 720 so as to discard such image data.

In various embodiments, when acquiring ultrasound images using the adapters 150, 170, the zero point of the ultrasound image (also called the apex in the case of sector imaging) may be set to be the depth of the non-attached surface 154, 174 of the respective adapters 150, 170 (instead of at the probe head 102). This may allow any image data collected from imaging depths shallower than the non-attached surface 154, 174 to be discarded. For example, referring again to FIG. 7, even in embodiments where the volume adjacent the pass-through volume 610 is provided with materials or mechanisms that will absorb and/or disperse the ultrasound energy to be masked (e.g., as may be dispersed by reflectors 720), there may nevertheless still be ultrasound energy that is not dispersed/absorbed but is reflected. By configuring the zero point of the ultrasound image to be the depth of the non-attached surface 154, 174, any reflected ultrasound energy emanating from such material and/or mechanism is unlikely to get incorrectly characterized as legitimate image data. Moreover, such configuration may help ensure that the reflected ultrasound energy (if any) resulting from the pass-through volumes 310, 610 of the adapters 150, 170, and/or the interface between the probe head 102 and the attached surfaces 120 of the adapters 150, 170, and/or the interface between the non-attached surface 154, 174 of the adapters 150, 170 and the skin all also do not get incorrectly characterized as legitimate image data.

The various embodiments discussed herein may facilitate imaging multiple patient areas using a single ultrasound transducer. For example, when used in a conventional context, a curvilinear probe 100 may be used to image the abdomen. However, with the attachment of the adapters 150, 170 discussed herein, the same curvilinear probe 100 may also be used to perform imaging that would typically require two additional probes (e.g., a traditional phased-array cardiac probe and a traditional linear probe). Put another way, the present embodiments may allow the single curvilinear probe 100 to serve the needs that would typically be served by three different ultrasound probes.

Various mechanisms may be used to configure the curvilinear probe 100 to employ the appropriate ultrasound sequence for the adapter 150, 170 that is attached. For example, in some embodiments, there may be a software setting that can be selected to configure the curvilinear probe 100 to modify its ultrasound sequence to activate in the manner discussed above with respect to FIG. 3 (e.g., if a cardiac adapter 150 is attached) or in the manner discussed above with respect to FIG. 6 (e.g., if a linear adapter 170 is attached).

Additionally or alternatively, electronic pins, connectors, or other like mechanism may be provided on the adapters 150, 170 so that they can couple with corresponding electronic components provided on the nosepiece 104. For example, when an electric connection is formed between such electronic components, the type of adapter 150, 170 can be communicated to the curvilinear probe 100 and the curvilinear probe 100 may alter its ultrasound sequence accordingly.

In some embodiments, the curvilinear probe 100 may be configured to automatically detect when an adapter 150, 170 is attached. For example, the curvilinear probe 100 and/or software controlling its operation may be configured to recognize pre-set image patterns generated from the reflections of the attached surfaces 120 of the adapters 150, 170 (as shown in FIG. 1). As the attached surfaces 120 of the adapters 150, 170 may produce distinct image patterns when ultrasound energy transmitted according to a conventional curvilinear ultrasound sequence is emitted from the probe head 102, such image patterns may be stored in memory so that upon initialization of the curvilinear probe 100, a test scan is performed. If the image data collected from the test scan matches any of the stored image patterns, then it may be determined that the adapter 150, 170 corresponding the stored image pattern is attached to the curvilinear probe 100 and the ultrasound sequence may be modified accordingly.

In some embodiments, to assist with generation and identification of such pre-set image patterns, the makeup of the non pass-through volume of an adapter 150, 170 (e.g., for the linear adapter 170, the volume corresponding to the non-hatched area shown in FIG. 8) may be constructed so as to provide a specific image signature that can be identified by the ultrasound device 100. For example, such volume may be created from a mixture of air and low acoustic impedance material such as acrylonitrile butadiene styrene (ABS), with the mixture being formed in a way that provides an image signature when ultrasound signals are projected onto it and reflected therefrom.

In another example, structures may be placed on the attached surfaces 120 (as shown in FIG. 1) beside the exposed surface of pass-through volumes 310, 610 discussed above. These structures can indirectly mate to the transducer array provided on the probe head 102 when the adapters 150, 170 are attached, so as to provide distinct image signatures that assist in identifying when a transducer adapter is attached and/or the type of transducer adapter attached. In various embodiments, the structures may be a metal plate or post, and the image signature may include the appearance of the structures when the structures are being imaged (e.g., using a traditional curvilinear ultrasound sequence). In various embodiments, the image signatures may include specific image artifacts that is created when imaging the structures (e.g., a comet tail or ring-down artifact).

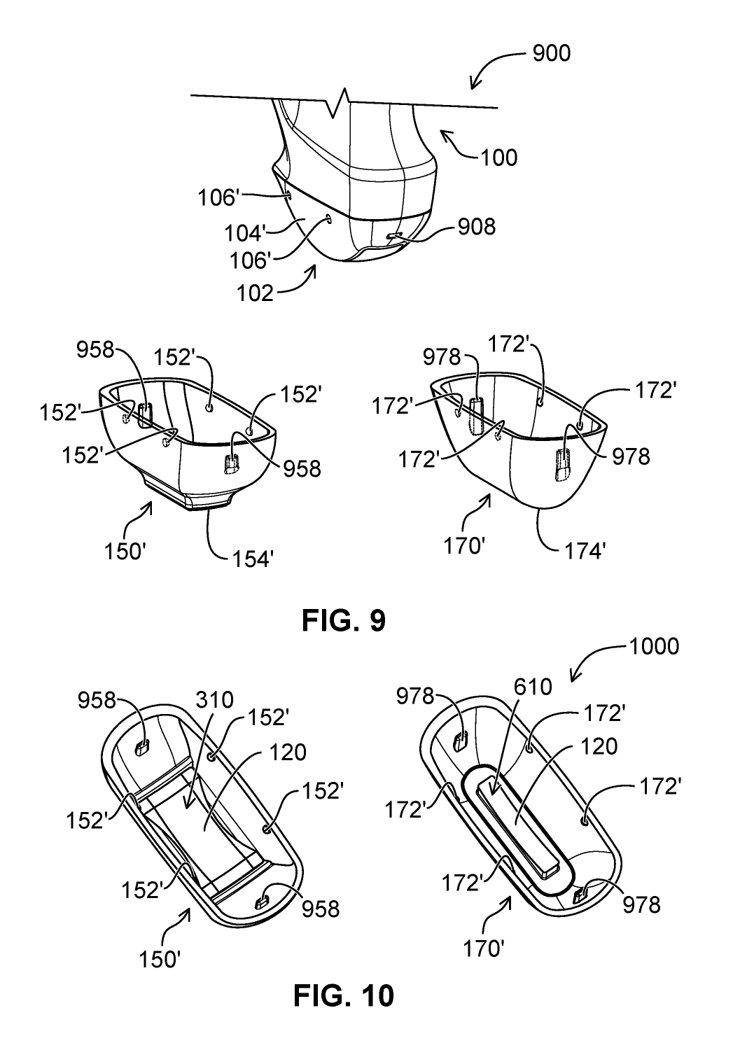

Referring to FIG. 9, shown there generally as 900 is a curvilinear ultrasound probe with example physical transducer adapters, in accordance with at least one embodiment of the present invention. Different transducer adapters 150′, 170′ may be attached to the probe head 102′. A nosepiece 104′ for holding the transducer array on the ultrasound probe 100 may be provided and configured with a mechanism to allow attachment of the adapters 150′, 170′. In the illustrated embodiment, nosepiece 104′ may be provided with divots 106′. Transducer adapters 150′, 170′ may be provided with corresponding protrusions 152′, 172′ so that a snap fit can be formed between transducer adapters 150′, 170′ and nosepiece 104′. In the example shown, hemispherical-shaped divots 106′ and corresponding protrusions 152′, 172′ are provided; however, any suitable shape may be used. Also, as illustrated, the divots 106′ are provided on the nosepiece 104′ and the corresponding protrusions 152′, 172′ are provided on the adapters 150′, 170′; however, in various embodiments, the divots may be provided on the adapters 150′, 170′, and the protrusions provided on the nosepiece 104′. In various embodiments, there may be different numbers of attachment mechanisms (e.g., divots and corresponding protrusions) other than what is shown.

As shown in FIG. 9, transducer adapters 150′, 170′ may be provided with one or more locator posts 958, 978 designed to be positioned against one or more locator slots 908 on nosepiece 106′. Locator posts 958, 978 may be configured to maintain a predetermined desired positioning of transducer adapters 150′, 170′ with respect to noise piece 104′. When the cardiac adapters 150′, 170′ attached, the locator posts 958, 978, abut against the slots 908. In addition to maintaining positioning, the interaction between the posts 958, 978 and the slots 908 may prevent the adapters from being pushed too far over the nosepiece 104′. In this manner, the posts 958, 978 and slots 908 may prevent damage of the transducer array of the ultrasound probe 100.

Similar to the example adapters 150, 170 with corresponding non-attached surfaces 154, 174 (also referred to as contact surfaces) shown in FIG. 1, non-attached surfaces 154′, 174′ form the distal end of respective pass-through volumes in the example adapters 150′, 170′ of FIG. 9. These contact surfaces 154′, 174′ may contact the object (e.g., anatomy) being imaged.

Referring to FIG. 10, shown there generally as 1000 is an interior perspective view of the cardiac adapter and linear adapter of FIG. 9, in accordance with at least one embodiment of the present invention. This view shows protrusions 152′, 172′ and posts 958, 978 used to attach the transducer adapters to ultrasound probe 100. This view also more clearly illustrates the components forming the pass-through volumes 310, 610 of the adapters 150′, 170′. As illustrated, each of the pass-through volumes 310, 610 has a respective proximal surface 120 that is configured to mate with the probe head 102 of ultrasound probe 100.

Generally, it is desirable for the distance between the contact surface 154′, 174′ and the proximal surface 120 of the adapters 150′, 170′ (e.g., the height of the pass-through volumes 310, 610) to be as short as a possible. By having the pass-through volumes 310, 610 have a short height, reverberation artifacts that result when ultrasound energy hits the attached (mating) surface 120 and the contact surface 154′, 174′ may be minimized. Also, since ultrasound energy emitted at predetermined frequencies may have a limited penetration depth, having the height of the pass-through volume 310, 610 be as short as possible may maximize the depth that the ultrasound signals can penetrate into the body and effectively image when the adapters 150′, 170′ are attached.

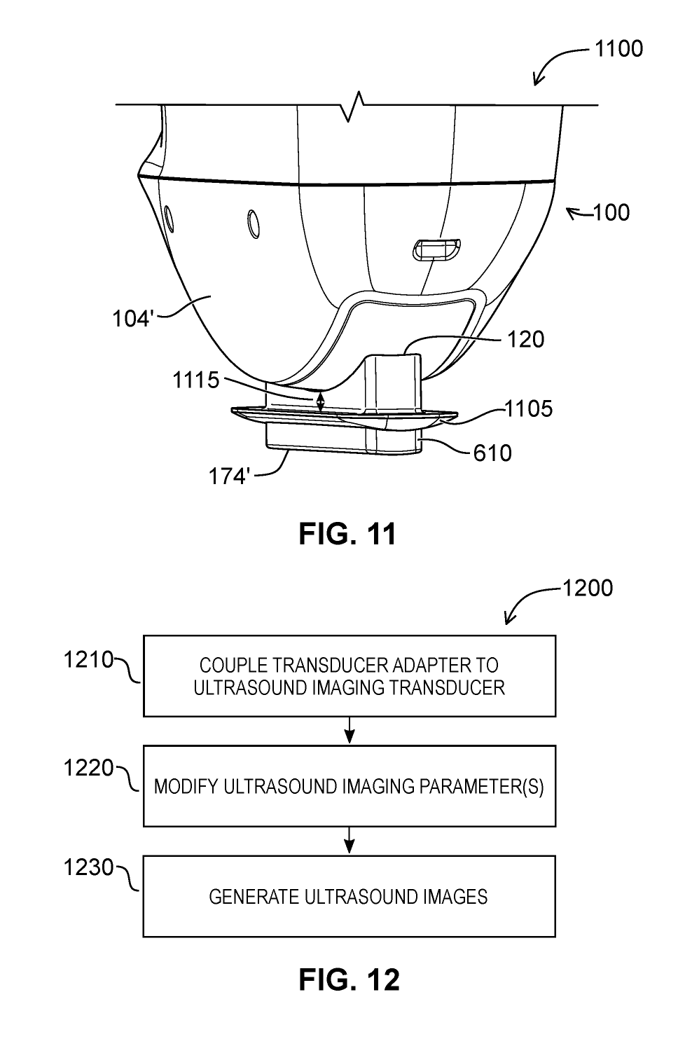

At the same time, there may be constraints as to how short the pass-through volumes 310, 610 can be. For example, in some embodiments, the pass-through volume 310, 610 may be separately manufactured (e.g., molded) out of a suitable acoustically-transparent material and, during manufacturing, adhered to the housing of an adapter 150′, 170′. Configuring the pass-through volume 310, 610 to be too short may make manufacturing difficult (e.g., because the molded material may not be sufficiently rigid to facilitate ease of adhesion to an interior surface of the housing of the adapters 150′, 170′). Also, as discussed below with respect to FIG. 11, for the linear adapter 170′, the height of the pass-through volume needs to be sufficiently high so as to prevent undesired acoustic coupling due to an acoustic coupling agent such as ultrasound gel.

Referring to FIG. 11, shown there generally as 1100 is a close-up perspective view of a pass-through volume component for the linear adapter in relation to an ultrasound probe, in accordance with at least one embodiment of the present invention. As noted, the pass-through volume 610 may be formed with a suitable acoustically-transparent material. In the embodiment of FIG. 11, the pass-through volume 610 is formed with flanges 1105 that can adhere to the interior surface of the housing for the linear adapter 170′ (not shown in FIG. 11) during assembly. FIG. 11 shows the pass-through volume component for the linear adapter 170′.

As discussed above, the elevation length of the pass-through volume 610 and contact surface 174′ for the linear adapter 170′ is shorter than the traditional elevation length of the transducer elements on a traditional curvilinear ultrasound probe 100. When the transducer adapters 170′ is attached to nose piece 104′ of ultrasound probe 100, the proximal surface 120 of the pass-through volume 610 may be offset a distance from the interior surface of the linear transducer adapter 170′. This offset 1115 distance may be chosen such that when an acoustic coupling agent (e.g., ultrasound gel) is interposed between the proximal surface 120 and the transducer, the pass-through volume 610 is acoustically coupled to the transducer while the volume adjacent the pass-through volume 610 is not acoustically coupled. For example, since using an acoustic coupling agent may allow for greater coupling between the transducer array and the pass-through volume 610, application of the acoustic coupling agent to the proximal surface 120 may improve imaging when using the transducer adapter 170′. However, once the transducer is positioned to be attached on the transducer adapter 170′, it is possible that the acoustic coupling agent spills or bleeds over the edges of proximal surface 120 to occupy the volume adjacent the pass-through volume 120. Configuring the offset distance 1115 to be sufficiently high may allow excess acoustic coupling agent to occupy a portion of the volume created by the offset distance 1115 while still maintaining an air gap between the transducer array and the flanges 1105 of the pass-through volume 610. This may limit acoustic coupling to only the pass-through volume 610 and prevent acoustic coupling between the transducer array and the volume adjacent the pass-through volume 610. Since such adjacent volume is intended to absorb/disperse ultrasound energy, the height 1115 may allow the desired slice thickness of the linear adapter 170′ to be achieved. This may also reduce unwanted image artifacts and improve image quality.

In this manner, the height of the offset distance 1115 may be configured to balance the desire to be as short as possible to minimize reverberation artifacts but still having enough height to prevent undesired acoustic coupling from the acoustic coupling agent. In various embodiments, the offset distance can be configured to be between 0-2.5 millimeters. However, other suitable offset distances may also be possible and are within the contemplation of the present embodiments.

Referring to FIG. 12, shown there generally as 1200 is a flow chart depicting a method for generating an ultrasound image using a transducer adapter, according to at least one embodiment of the present invention. The method may be performed by ultrasound probe 100. Additionally or alternatively, the method or parts thereof may be performed by a secondary device controlling the ultrasound probe, such as a multi-use electronic display device (not shown). In discussing the method of FIG. 12, reference will simultaneously be made to the elements of FIGS. 1, 3, and 6 discussed above.

At 1210, a transducer adapter 150, 170 is coupled to ultrasound imaging transducer 100. As described herein, the transducer adapter 150, 170 may be coupled directly to the ultrasound probe 100. As discussed, a coupling agent (e.g., ultrasound gel) may be interposed between transducer adapter 150, 170 and ultrasound probe 100 to improve acoustic transmission between the two. The proximal surface of the pass-through volume 310, 610 can be mated with the probe's transducer surface.

The geometry of the probe transducer surface may define a native footprint corresponding to its curvature and surface area. The distal surface of the transducer adapter 150, 170's pass-through volume 310, 610 may define an adapted footprint, representing a curvature and surface area different from the native footprint. Different types of probes 100 may have different footprints. For example, a cardiac probe may have a small footprint for cardiac imaging and a linear probe may have a linear, substantially planar footprint.

Different adapter types may be defined that mate with a particular footprint on its proximal end, and have a different adapted footprint on its distal end. For example, as discussed above, a cardiac adapter 150 for a curvilinear probe 100 may have a curvilinear proximal surface 120 to mate with the curvilinear transducer 100, and a cardiac footprint on the distal surface 154 to contact an object being imaged. Similarly, a linear adapter 170 for a curvilinear probe 100 may have a curvilinear proximal surface 120 to mate with the curvilinear transducer 100, and a linear footprint on the distal surface 174 to contact an object being imaged.

At 1220, at least one ultrasound imaging parameter is modified. The imaging parameters may be modified so as to result in the ultrasound energy being emitted from (and received by) the adapted footprint of the distal contact surfaces 154, 174 of the adapter 150, 170 in a similar manner as the ultrasound energy would be emitted from and received by an ultrasound transducer with a native footprint that matches the adapted footprint.

For example, time delay and aperture may be modified as described in FIGS. 3 and 6 so that the ultrasound signals emitted by the ultrasound probe transducer are steered in a direction that is away from normal to the native footprint of the ultrasound transducer array. Whereas ultrasound signals generated from a curvilinear probe are traditionally transmitted and received sequentially from different apertures in directions normal to the probe's native footprint (e.g., in non-steered directions), FIGS. 3 and 6 above show how the ultrasound signals can be steered to provide ultrasound signals that are suitable for the contact surfaces 154, 174 of the adapted footprints. For example, as shown in FIG. 3, the apertures and time delays can be modified to steer ultrasound signals so that they are emitted and received in a phased manner similar to ultrasound signals traditionally emitted from a cardiac probe. Similarly, as shown in FIG. 6, apertures and time delays can be configured so that ultrasound signals emitted from the ultrasound probe are not normal to the transducer array (at the aperture where the signals are being emitted from); but they exit the contact surface 174 in directions that is normal to the substantially planar footprint of the linear adapter 174 so as to yield parallel scanlines similar to those generated using a conventional linear ultrasound probe.

Depending on the transducer type, one or more of time delay, sequence, steering angle, transmit aperture size, transmit aperture location, receive aperture size, receive aperture location, and image zero point may be modified.

In some embodiments, the ultrasound imaging parameters can be modified based on the transducer adapter type. The adapter type may be considered to be based on at least one of a geometry of: the proximal end configured to mate to the ultrasound imaging transducer, and a geometry of the contact surface. Two example adapters have been discussed herein: an cardiac-type adapter 150 and a linear-type adapter 170.

The transducer adapter type may be determined in several different ways. For example, an adapter type may be determined manually through input from the operator. This input may be through a selection made in software, or by actuating a physical switch on the ultrasound probe. For example, an operator may specify or choose an adapter type from a list of options in a user interface.

Additionally or alternatively, the adapter type may be determined automatically by the ultrasound probe. For example, the probe may include a sensor and the adapter may include a sensible element so that the probe can determine the adapter type. In various embodiments, this sensor could be based on radiofrequency identification (RFID), near-field communication (NFC), and/or other conventionally known or future developed sensing technologies.

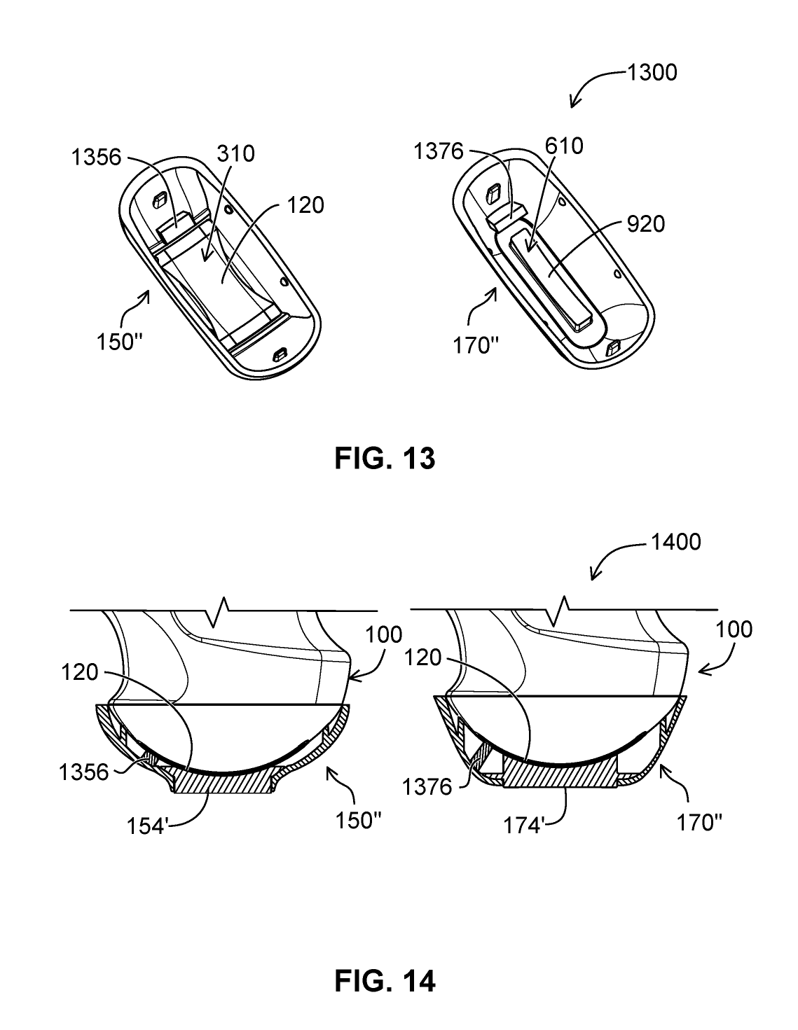

Additionally or alternatively, the transducer adapter may include an image signature feature that enables the probe to detect adapter type using ultrasound image as described above and with reference to FIG. 13 below.

Referring back to FIG. 12, at 1230, an ultrasound image can be generated by ultrasound probe 100. Depending on the adapter type, the generated ultrasound image may have different shapes. For example, with a linear-type adapter 170, the image will be rectangular. In another example, the ultrasound image generated by the ultrasound probe with a cardiac-type adapter 150 attached will be sector-shaped.

Referring to FIG. 13, shown there generally as 1300, is an interior perspective view of an alternative embodiment of the cardiac adapter and linear adapter, in accordance with at least one embodiment of the present invention. As described above, the attached surface 120 is configured to mate to the transducer array of ultrasound probe 100. In this embodiment, transducer adapter 150″, 170″ includes an image signature feature 1356, 1376. As described above, image signature feature 1356, 1376 may be configured to produce a characteristic image signature when transducer adapter 150″, 170″ is coupled to ultrasound probe 100 and an ultrasound image is generated. The characteristic image signature produced by image signature feature 1356, 1376 may be used by ultrasound probe 100 to determine the adapter type of the attached transducer adapter 150″, 170″.

As described above, image signature feature 1356, 1376 may be constructed of the same material as the pass-through volume 310, 610, and/or may be constructed of a material that yields a characteristic signature, and/or may be constructed of the same material as the housing of the transducer adapter 150″, 170″. In various embodiments, the ultrasound probe 100 may differentiate between two different adapter types based on one or more of the following characteristics of the image signature feature 1356, 1376: echogenicity, feature width, and feature depth.

Referring to FIG. 14, shown there generally as 1400, is a cross-sectional view of the cardiac adapter and linear adapter of FIG. 13, in accordance with at least one embodiment of the present invention. As shown, the attached surface 120 is mated to the transducer array of ultrasound probe 100. Non-attached surfaces 154′, 174′ form the distal end of the pass-through volumes similar to what was described with reference to the embodiments of FIG. 9.

FIG. 14 shows a cross sectional view of image signature feature 1356, 1376. The image signature feature 1356, 1376 is also configured to mate with the transducer 100 so that ultrasound signals may be projected to and reflected from it for the purpose of identifying the image signature features 1356, 1376 on an ultrasound image. In various embodiments, there may be a gap between where attached surface 120 couples to the transducer 100 and where image signature feature 1356, 1376 couples to the transducer array so that two separate sets of transducer elements are connected when the adapters 150″, 170″ are attached. These separate transducer elements sets may be used by the probe to determine adapter type.

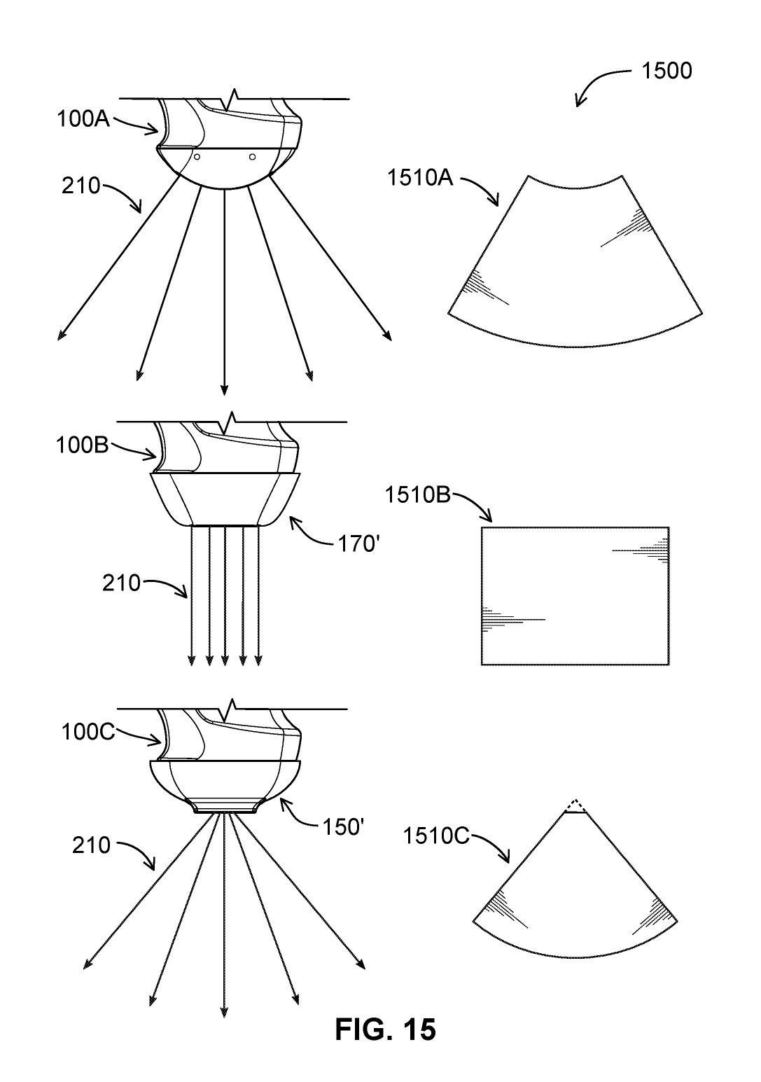

Referring to FIG. 15, shown there generally as 1500, is a diagram that shows the different imaging configurations of the ultrasound imaging probe and example cardiac and linear transducer adapters, in accordance with at least one embodiment of the present invention. A single ultrasound probe 100 may be used in several different configurations 100A, 100B, 100C to image different parts of a patient or generate different types of ultrasound images.

Configuration 100A shows ultrasound probe 100 with no transducer adapter attached. It may be operated in a conventionally-known manner to emit ultrasound signals 210 and generate an image 1510A like that which is typically produced by a sequential curvilinear probe.

Configuration 100B shows ultrasound probe 100 with a linear adapter 170′ attached. The substantially planar contact surface area of the adapted footprint for the linear adapter 170′ may enable the curvilinear ultrasound probe to couple more effectively to a substantially planar object that would otherwise require large amounts of coupling agent (e.g., ultrasound gel) or a forceful application of the ultrasound probe 100 onto the surface being imaged. Reducing the force required to effectively acoustically couple to the object being imaged may improve operator ergonomics and/or reduce tissue deformation that negatively affects image quality.

As noted, configuration 100B may emit ultrasound signals 210 to generate a rectangular image 1510B similar to a conventional ultrasound image generated by an ultrasound probe having a linear transducer geometry. As described with reference to FIG. 6-8 above, the steering of the ultrasound signals and reduced elevational length may generate an ultrasound image that shares other characteristics of a conventional linear ultrasound image, including consistent lateral resolution at various imaging depth, and improved elevational resolution.

Configuration 100C show ultrasound probe 100 with a cardiac adapter 150′ attached. The smaller surface area of the adapted footprint of cardiac adapter 150′ may allow the operator to position the probe in a better position for imaging. For example, when imaging between the ribs, the smaller surface area of the adapted footprint may allow the contact surface to press further into the tissue between the patient's ribs, enabling a better image to be obtained. The smaller surface area of the adapted footprint may also mean that the operator need to exert less force, leading to less operator fatigue and better ergonomics.

As described with reference to FIG. 2-5 above, configuration 100C may emit ultrasound signals 210 in a phased manner so as to generate a fan-shaped (e.g., sector) image 1510C similar to the image generated by an ultrasound probe with a phased array transducer geometry.

As discussed above with respect to FIG. 3, when the cardiac adapter 150 is attached, ultrasound signals 210 can be configured to exit the contact surface of the cardiac adapter so that the apex of the resultant sector image is at the non-attached surface 154 in the direction. However, in various embodiments, the apex of the sector image can be configured to be slightly higher (e.g., at the attached surface 120 of the cardiac adapter 150, as shown in FIG. 1). This may result in a sector image 1510C that is slightly clipped at the top (e.g., show dotted lines in FIG. 15). This type of clipped image may allow a substantial portion of a sector image to be provided and may be generated, for example, if the cardiac adapter 150 is attached to an ultrasound transducer 100 that is imaging in a sector imaging mode that is described in Applicant's Virtual Phased-array Application.

Using the embodiments described herein, an operator may quickly switch between configurations 100A-C by detaching and attaching transducer adapters 150′, 170′ to an ultrasound probe 100 as desired. While the description above refers to using ultrasound probe 100 with cardiac adapter 150′ and linear adapter 170′, persons skilled in the art will recognize that just one type of transducer adapter may be used, or different types of transducer adapter may be used.

While a number of exemplary aspects and embodiments have been discussed above, those of skill in the art will recognize that may be certain modifications, permutations, additions and sub-combinations thereof. While the above description contains many details of example embodiments, these should not be construed as essential limitations on the scope of any embodiment. Many other ramifications and variations are possible within the teachings of the various embodiments.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.
Unless the context clearly requires otherwise, throughout the description and the claims:

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.
Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.
For example, while processes or blocks are presented in a given order herein, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.
The invention may also be provided in the form of a program product. The program product may comprise any non-transitory medium which carries a set of computer-readable instructions which, when executed by a data processor (e.g., in a controller and/or ultrasound processor in an ultrasound machine), cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.
Where a component (e.g. an adapter, a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.
Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.
It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. An ultrasound imaging transducer assembly, comprising:
an ultrasound imaging transducer having transducer elements configured in a curved geometry; and
a transducer adapter for coupling to the ultrasound imaging transducer, wherein the transducer adapter comprises a pass-through volume for permitting ultrasound energy to be transmitted through the transducer adapter, the pass-through volume comprising:
a proximal surface for mating to the transducer, the proximal surface having a curvature corresponding to the curved geometry of the transducer elements; and
a distal surface at a distal end of the transducer assembly, the distal surface having a geometry that is substantially planar.

2. The ultrasound imaging transducer assembly of claim 1, wherein the ultrasound imaging transducer is configured to determine an adapter type of the transducer adapter.

3. The ultrasound imaging transducer assembly of claim 2, wherein the ultrasound imaging transducer further comprises a sensor for determining the adapter type of the transducer adapter.

4. The ultrasound imaging transducer assembly of claim 3, wherein the transducer adapter includes a radio frequency identification (RFID) tag and the sensor comprises a radio frequency identification (RFID) sensor.

5. The ultrasound imaging transducer assembly of claim 1, wherein the transducer adapter further comprises an image signature feature readable by the ultrasound imaging transducer when the transducer adapter is coupled to the ultrasound imaging transducer.

6. The ultrasound imaging transducer assembly of claim 1, wherein the pass-through volume comprises at least one of agar, agarose, Aqualene™, silicone, polyvinyl alcohol, polyvinyl alcohol gel, polyacrylamide gel, open porosity foam, gelatin gel, oil gel, polyurethane gel, epoxy plastisol, silicon rubber, swollen segmented polyurethane gel (S-SPUG), urethane polymer, tofu, magnesium silicate, and Zerdine™.

7. The ultrasound imaging transducer assembly of claim 1, wherein the ultrasound transducer elements have a first elevational length and the proximal surface has a second elevational length, different from the first elevational length.

8. A method of generating ultrasound images with an ultrasound imaging transducer, the ultrasound imaging transducer having a native footprint, the method comprising:
coupling a transducer adapter to the ultrasound imaging transducer, wherein the transducer adapter has a proximal end configured to mate to the ultrasound imaging transducer and a distal end that defines a contact surface, wherein the contact surface corresponds to an adapted footprint different from the native footprint;
modifying at least one imaging parameter so that, during imaging, ultrasound signals emitted from the ultrasound imaging transducer travels through the transducer adapter and exits the contact surface, and the exiting ultrasound signals have one or more characteristics substantially similar to other ultrasound signals emitted from another ultrasound imaging transducer having the adapted footprint as its native footprint; and
generating the ultrasound images with the modified at least one imaging parameter.

9. The method of claim 8, wherein after the transducer adapter is coupled, the modified at least one imaging parameter causes the at least one of the ultrasound signals emitted from the ultrasound imaging transducer to be steered in a direction away from normal to the native footprint of the ultrasound imaging transducer.

10. The method of claim 9, wherein prior to the transducer adapter being coupled, during imaging, the ultrasound imaging transducer emits non-steered ultrasound signals.

11. The method of claim 9, wherein the modified at least one imaging parameter comprises at least one of time delay and aperture, to cause the at least one of the ultrasound signals to be steered in the direction away from normal to the native footprint of the ultrasound imaging transducer.

12. The method of claim 9, wherein the adapted footprint comprises a cardiac footprint, and wherein the exiting ultrasound signals are steered in respective different directions so that a substantial portion of a sector image is generated.

13. The method of claim 9, wherein the adapted footprint comprises a linear footprint, and wherein the steered at least one of the ultrasound signals emitted from the ultrasound imaging transducer results in the exiting ultrasound signals being projected orthogonally to the contact surface.

14. The method of claim 13, wherein the exiting ultrasound signals comprise parallel ultrasound signals so that a rectangular image is generated.

15. The method of claim 8, further comprising, after coupling the transducer adapter:
determining an adapter type of the transducer adapter, wherein the adapter type is based on at least one of a geometry of: the proximal end configured to mate to the ultrasound imaging transducer, and a geometry of the contact surface.

16. The method of claim 15, wherein the modifying of the at least one imaging parameter is based at least in part on the adapter type.

17. The method of claim 16, wherein the ultrasound imaging transducer further comprises a sensor and wherein the determining the adapter type of the transducer adapter comprises:
sensing the adapter type of the transducer adapter using the sensor.

18. The method of claim 15, wherein an image signature feature is provided on the transducer adapter that is readable when the transducer adapter is coupled to ultrasound imaging transducer, and the determining the adapter type of the transducer adapter comprises:
generating an ultrasound image; and
identifying the image signature feature on the ultrasound image to determine the adapter type of the transducer adapter.

19. A transducer adapter for coupling to an ultrasound imaging transducer with a native footprint, the transducer adapter comprising:
a proximal end configured to mate to the ultrasound imaging transducer; and
a distal end that defines a contact surface, wherein the contact surface corresponds to an adapted footprint different from the native footprint;
wherein when the transducer is coupled to the ultrasound imaging transducer, ultrasound signals emitted from the ultrasound imaging transducer travels through the transducer adapter and exits the contact surface, and the exiting ultrasound signals have one or more characteristics that are substantially similar to ultrasound signals emitted from another ultrasound imaging transducer having the adapted footprint as its native footprint.

20. The transducer adapter of claim 19, wherein the transducer adapter comprises a pass-through volume for permitting the ultrasound signals to be transmitted through the transducer adapter.

21. The transducer adapter of claim 20, wherein the pass-through volume comprises at least one of agar, agarose, Aqualene™, silicone, polyvinyl alcohol, polyvinyl alcohol gel, polyacrylamide gel, open porosity foam, gelatin gel, oil gel, polyurethane gel, epoxy plastisol, silicon rubber, swollen segmented polyurethane gel (S-SPUG), urethane polymer, tofu, magnesium silicate, and Zerdine™.

22. The transducer adapter of claim 19, wherein the transducer adapter further comprises an image signature feature readable by the ultrasound imaging transducer when the transducer adapter is coupled to the ultrasound imaging transducer.
